(19) United States
(12) Patent Application Publication
(10) Pub. No.: US 2025/0026776 A1
(43) Pub. Date: Jan. 23, 2025

(54) Method for Simultaneously Separating and Purifying Two Galloylated Myricitrins from Morella rubra Leaves, and Use therefor

(71) Applicant: Zhejiang University, Hangzhou (CN)

(72) Inventors: Xian LI, Hangzhou (CN); Yilong LIU, Hangzhou (CN); Xiaoyong ZHAO, Hangzhou (CN); Chongde SUN, Hangzhou (CN); Changjie XU, Hangzhou (CN); Kunsong CHEN, Hangzhou (CN)

(21) Appl. No.: 18/908,699

(22) Filed: Oct. 7, 2024

Related U.S. Application Data
(63) Continuation of application No. PCT/CN2023/083008, filed on Mar. 22, 2023.

(30) Foreign Application Priority Data
Apr. 8, 2022 (CN) 202210368387.0
Oct. 26, 2022 (CN) 202211327485.6

(51) Int. Cl. C07H 17/07; C07H 1/08

(52) U.S. Cl. CPC C07H 17/07; C07H 1/08

(57) ABSTRACT

A method for simultaneously separating and purifying myricetin-3-O-(2″-O-galloyl)-a-L-rhamnoside and myricetin-3-O-(4″-O-galloyl)-a-L-rhamnoside from Bayberry leaves, and the use of the compounds prepared by the method. The method includes: (1) alcohol extraction and concentration: mixing Bayberry leaves with an alcohol solution; (2) solid-phase extraction column adsorption: injecting the crude Bayberry leaf flavonol alcohol extract into a solid-phase extraction column; washing with a mobile phase for the first gradient elution; (3) preparative liquid chromatography purification: using a solid-phase chromatography column; wherein the solid-phase extraction powder obtained from step (2) is subjected to a second gradient elution with a mobile phase and then processed to obtain the target products: myricetin-3-O-(2″-galloyl)-α-L-rhamnoside monomer and myricetin-3-O-(4″-galloyl)-α-L-rhamnoside monomer.

### CROSS REFERENCE TO RELATED APPLICATIONS

This application is a continuation of International Patent Application No. PCT/CN2023/083008, filed on 22 Mar. 2023, entitled “Method for Simultaneously Separating and Purifying Two Galloylated Myricitrins from Morella rubra Leaves, and Use therefor,” which claims foreign priority of Chinese Patent Application No. 202210368387.0, filed 8 Apr. 2022, and Chinese Patent Application No. 202211327485.6, filed 26 Oct. 2022 in the China National Intellectual Property Administration (CNIPA), the entire contents of which are hereby incorporated by reference in their entireties.

### TECHNICAL FIELD

This invention relates to the field of separation and purification of natural products, specifically to a method and use for simultaneously separating and purifying myricetin-3-O-(2″-galloyl)-α-L-rhamnoside and myricetin-3-O-(4″-galloyl)-α-L-rhamnoside from bayberry leaves.

### BACKGROUND

In recent decades, with changes in lifestyle and living conditions, the incidence of diabetes and its complications has risen sharply, becoming a serious global health issue. Diabetes is a metabolic disease characterized by high blood sugar, and long-term hyperglycemia can lead to metabolic disorders in the body, causing various metabolic syndromes such as obesity, hyperlipidemia, and hypertension. The high prevalence of diabetes poses a severe social and economic burden on countries, especially middle- and low-income nations. Therefore, effective prevention and treatment measures are urgently needed to control the rapid development of diabetes and other metabolic syndromes.

α-Glucosidase inhibitors have been recommended as first-line drugs for the treatment of diabetes and are an effective method for preventing related complications, improving insulin resistance, and effectively preventing and treating diabetes, obesity, and other metabolic syndromes. α-Glucosidase is a membrane-bound enzyme in the glycoside hydrolase family GH31, mainly found in the microvillus membrane brush border cells of the small intestine villi. After eating, α-glucosidase can hydrolyze carbohydrates in food into glucose, which, when absorbed, enters the bloodstream and causes blood sugar levels to rise. Diabetic patients, due to islet dysfunction, have impaired blood sugar regulation ability and cannot effectively control the rapid increase in blood sugar levels after meals. α-Glucosidase inhibitors effectively control the rapid increase in postprandial blood sugar by delaying the decomposition and absorption of carbohydrates and glucose release, thereby improving insulin resistance and inhibiting fat synthesis. At present, the commonly used α-glucosidase inhibitors in clinical practice mainly include acarbose, voglibose, and miglitol, but these drugs often cause gastrointestinal adverse reactions, such as bloating, flatulence, and diarrhea. Therefore, finding new α-glucosidase inhibitors with strong inhibitory activity and lower toxicity is of great significance for the prevention and treatment of diabetes, obesity, and other metabolic syndromes.

In recent years, it has been found that various natural flavonoid compounds have a strong inhibitory effect on α-glucosidase, attracting widespread attention from domestic and foreign scholars. Flavonoids generally refer to a series of substances connected by three carbon atoms (ring C) of two benzene rings (ring A and ring B), that is, a general term for a class of compounds with a C6-C3-C6 structure. According to the oxidation degree of ring C and the connection position of ring B and other structural characteristics, flavonoids can be further divided into flavonol, anthocyanin, flavone, flavanone, and flavanol subclasses. In a comparative study of the α-glucosidase inhibitory activity of 27 dietary flavonoids, myricetin was identified as the strongest inhibitor (Jia Y, et al., Journal of Agricultural and Food Chemistry 67(37): 10521-10533 (2019)). Similarly, in another report on the α-glucosidase inhibitory activity of 15 types of flavonoids, myricetin also showed the strongest inhibitory effect, followed by robinetin and quercetin (He C., et al., Foods 8(9): 355 (2019)), all of which belong to the flavonol subclass. This indicates that myricetin and other flavonol compounds may be α-glucosidase inhibitors with great development potential.

Bayberry leaves are a natural resource rich in flavonols such as myricetin. The bayberry tree is evergreen with lush branches and leaves. To avoid the impact of apical dominance on fruit setting and to promote continuous, high-quality, and high-yield production of bayberries, fruit farmers need to prune the bayberry trees in both spring and autumn, resulting in a large amount of waste bayberry leaves. These leaves are usually used as firewood for burning, increasing carbon emissions and causing environmental pollution. Ancient medical books record that bayberry leaves are bitter, slightly spicy, and warm in nature, and can be used to treat diseases such as diarrhea, jaundice, lymph node tuberculosis, and chronic pharyngitis. In recent years, a large number of studies have shown that bayberry leaf extracts have good free radical scavenging, anti-inflammatory, and bacteriostatic activities. Therefore, the development and utilization of bayberry branches and leaves can not only solve the environmental pollution problems brought by waste disposal but also turn waste into treasure and increase income, benefiting human health. Bayberry leaves specifically accumulate myricetin, with a content of more than 10 mg/g fresh weight. However, in the leaves, myricetin usually exists in the form of derivatives modified by glycosylation and galloylation. However, due to the similar structures and small polarity differences of these flavonol derivatives, the separation of high-purity monomers is very difficult, and their structures cannot be accurately characterized, which greatly limits the exploration of the pharmacological activities of bayberry leaves and their further development and utilization.

For example, Zhang et al. identified flavonol compounds in bayberry leaves by LC-MS, but only inferred their possible structures based on the secondary fragment ion map without precise structural analysis (Zhang, Y, PLoS One 11(12): e0167484 (2016)); Chen Ping et al. (“Screening and Separation and Purification of Bactericidal Components in Bayberry Leaves”, Food Science and Technology, 2011, 36(2): 189-192) separated and purified flavonoid crude extracts from the alcohol extract of bayberry leaves by multi-step organic solvent extraction, macroporous adsorption resin column chromatography, and gel column chromatography, but failed to purify the relatively pure flavonol monomers; Kim, H. H., et al. (Archives of Pharmacal Research, 36(12): 1533-1540 (2013)) separated and purified flavonols from bayberry leaves, and the crude extract of bayberry leaves was repeatedly column chromatographed, followed by reversed-phase medium-pressure liquid chromatography to obtain flavonol monomers, but the extraction and purification process was complicated, the extraction efficiency was low, and it limited the comprehensive utilization of bayberry leaves.

### SUMMARY OF THE INVENTION

To solve the above technical problems, this invention provides a method and use for simultaneously separating and purifying myricetin-3-O-(2″-galloyl)-α-L-rhamnoside and myricetin-3-O-(4″-galloyl)-α-L-rhamnoside from bayberry leaves.

Based on this, the inventors of this invention have established a separation and purification system combined with alcohol extraction and concentration, solid-phase extraction column purification, and preparative liquid chromatography. For the first time, myricetin-3-O-(4″-O-galloyl)-α-L-rhamnoside (CAS number: 85541-03-3) was separated from bayberry leaf blades, and it was found that this compound (IC₅₀=15.61 μM) has significantly better α-glucosidase inhibitory activity than the positive control drug acarbose (IC₅₀=369.15 μM) and myricetin (IC₅₀=1.77 μM). Secondly, myricetin-3-O-(2″-O-galloyl)-α-L-rhamnoside (CAS number: 56939-52-7) was also separated, and it was found that it can also significantly inhibit α-glucosidase activity (IC₅₀=1.32 μM). These two compounds are isomers, and due to their similar structures and close polarity, the difficulty of separation and purification is great. The latest literature (da Silva, G. L., et al., Natural Product Research 22:1-5 (2021)) found that these two compounds are contained in the leaves of Syzygium aromaticum, but they have not been able to achieve the separation and preparation of the two, so they only measured the activity of the mixture containing these two substances. This invention has successfully established a rapid and efficient purification system for these two compounds, which can simultaneously prepare high-purity (purity above 98%) myricetin-3-O-(4″-galloyl)-α-L-rhamnoside and myricetin-3-O-(2″-galloyl)-α-L-rhamnoside monomers. Activity tests conducted on the purified flavonol monomers revealed their excellent α-glucosidase inhibitory activity, indicating their significant potential for development as α-glucosidase inhibitors for the prevention and treatment of metabolic syndromes such as diabetes and obesity. This discovery is of great importance for further exploration of functional components in bayberry leaves, investigation of their pharmacological activities, and enhancement of the added value of the bayberry industry.

The invention employs the following technical scheme:

A method for simultaneously separating and purifying myricetin-3-O-(2″-galloyl)-α-L-rhamnoside and myricetin-3-O-(4″-galloyl)-α-L-rhamnoside from bayberry leaves, which includes: (1) Alcohol Extraction and Concentration: Mix bayberry leaves with an alcohol solution, extract them ultrasonically, filter and collect the filtrate. Remove the alcohol from the filtrate and concentrate it to obtain a crude bayberry leaf flavonol extract. The volume percentage of the alcohol solution is between 50-100%.

(2) Solid-phase extraction column adsorption: The crude bayberry leaf flavonol extract is injected into a solid-phase extraction column, and the first gradient elution is performed with a mobile phase. The collected eluent is then processed to obtain a solid-phase extraction powder rich in myricetin-3-O-(2″-galloyl)-α-L-rhamnoside and myricetin-3-O-(4″-galloyl)-α-L-rhamnoside. Preferably, the solid-phase extraction column is a C18 solid-phase extraction column. The first gradient elution process involves: first eluting with an alcohol solution with a volume percentage below 40% for the initial elution, followed by a second elution with an alcohol solution with a volume percentage between 40% and 60%, collecting the eluent after the second elution; the volume percentage concentration of the alcohol solution for the first elution is above 20%.

Preferably, a 30% alcohol solution by volume is used for the first elution.

Preferably, a 40% alcohol solution by volume is used for the second elution.

(3) Preparative liquid chromatography purification: A solid-phase chromatography column is used to perform a second gradient elution with the mobile phase on the solid-phase extraction powder obtained from step (2), followed by post-processing to separately obtain the target products: myricetin-3-O-(2″-galloyl)-α-L-rhamnoside monomer and myricetin-3-O-(4″-galloyl)-α-L-rhamnoside monomer. Preferably, the solid-phase chromatography column is a C18 solid-phase chromatography column.

The mobile phase: Phase A consists of a formic acid-water solution with a volume percentage of 0.05% to 5%, and a trifluoroacetic acid-water solution with a volume percentage of 0.05% to 5%. Phase B consists of an acetonitrile-water solution with a volume percentage of 40% to 60%, and an acid-acetonitrile-water solution with a volume percentage of 40% to 60%, where the volume percentage of the acid is 0.05% to 5%, and the acid is selected from formic acid and trifluoroacetic acid.

The second gradient elution process involves: increasing the volume percentage of Phase B from 20% to 60% within 0-10 minutes, from 60% to 90% within 10-30 minutes, and from 90% to 100% within 30-35 minutes, and then decreasing from 100% to 20% within 35-40 minutes, collecting the target products separately.

Preferably, Phase A consists of a formic acid-water solution with a volume percentage of 0.1% to 3%.

Preferably, in step (3), a preparative liquid chromatography column SunFire™ C18 OBM™ column (5 μm, 19×250 mm) is used, and during the second gradient elution, the eluates at 27-28.5 minutes and 28.5-30 minutes are collected separately. More preferably, the column temperature is room temperature, and the flow rate is 3-6 mL/min.

Preferably, the solid-phase extraction powder obtained from step (2) is dissolved in methanol to achieve a concentration of 100-200 mg/mL. This solution is then injected into the preparative liquid chromatography for purification, with a single injection volume ranging from 50 to 300 μL.

The alcohol solution mentioned refers to an aqueous solution of alcohol, where the alcohol is selected from methanol or ethanol.

Preferably, the volume percentage of the alcohol solution is 80%.

Preferably, the ultrasound extraction time is between 30 to 60 minutes.

Preferably, the mass-to-volume ratio of bayberry leaves to methanol solution is between 1:5 and 1:20. More preferably, this ratio is 1:10.

Preferably, the post-treatment involves reduced pressure concentration and freeze-drying. More preferably, the reduced pressure concentration is conducted under vacuum rotary evaporation at a temperature range of 37 to 50° C.

Preferably, the purity of the myricetin-3-O-(2″-galloyl)-α-L-rhamnoside monomer and the myricetin-3-O-(4″-galloyl)-α-L-rhamnoside monomer is above 98%; more preferably, the purity is above 99%.

Preferably, in step (1), the filtrate is subjected to vacuum rotary evaporation at 37-50° C. to remove the alcohol and concentrate the solution, resulting in a crude bayberry leaf flavonol extract.

Preferably, in step (1), the process of collecting the filtrate is repeated 2 to 4 times, and the filtrates from 2 to 4 collections are combined.

Preferably, in step (2), the solid-phase extraction column is eluted using a gradient elution process, which specifically involves:

Injecting the crude bayberry leaf flavonol extract into the solid-phase extraction column, initially flushing the column with deionized water, followed by gradient elution with a mobile phase, and then concentrating the collected eluent under vacuum rotary evaporation at 37-45° C. to obtain the solid-phase extraction powder enriched with the target products.

It is noted that the purpose of the first elution with an alcohol solution below 40% is to remove impurities such as saccharic acid and other non-target flavonols like myricitrin; if the concentration of the alcohol solution for the first elution is below 20%, the flavonols will not be eluted effectively.

Preferably, in step (2), the adsorption on the solid-phase extraction column is as follows:

After activating the C18 Sep-Pak® solid-phase extraction column, load 4.5 bed volumes (BV) of the bayberry leaf extract onto each column; wash away saccharic acid with 4 BV of deionized water; then elute with 10 BV of a 30% methanol solution to remove some impurities and other non-target flavonols; subsequently, elute with 4 BV of a 40% methanol solution, collect the 40% fraction eluent, and dry it under vacuum rotary evaporation at 37-50° C. to obtain the solid-phase extraction powder enriched with myricetin-3-O-(2″-galloyl)-α-L-rhamnoside and myricetin-3-O-(4″-galloyl)-α-L-rhamnoside.

Preferably, in step (3), Phase B is selected from a 50% acetonitrile-water solution and a 50% acid-acetonitrile-water solution, with the volume percentage of the acid being 0.1% to 5%, and the acid is selected from formic acid and trifluoroacetic acid; more preferably, the volume percentage of the acid is between 0.1% and 3%.

Preferably, in step (3), the preparative liquid chromatography purification is as follows:

Use a preparative liquid chromatography column SunFire™ C18 OBM™ (5 m, 19×250 mm), with the mobile phase consisting of: Phase A, a 0.1% formic acid-water solution, and Phase B, a 50% acetonitrile-water solution (containing 0.1% formic acid); the column temperature is ambient, and the flow rate is 3-6 mL/min.

The solid-phase extraction powder obtained from step (2) is dissolved in methanol to a concentration of 100-200 mg/mL. This solution is then injected into the preparative liquid chromatography for purification, with a single injection volume of 50-300 μL. The eluates collected separately during 27-28.5 minutes and 28.5-30 minutes are combined, and the combined eluate rich in pure target products is subjected to reduced pressure concentration and freeze-drying to yield high-purity myricetin-3-O-(2″-galloyl)-α-L-rhamnoside monomer and myricetin-3-O-(4″-galloyl)-α-L-rhamnoside monomer.

The alcohol solution refers to methanol or ethanol. Research has found that acetone extracts have a low concentration of target products and cannot fully extract the target products from the samples. Solvents such as ethyl acetate and petroleum ether fail to extract the target products (myricetin-3-O-(2″-galloyl)-α-L-rhamnoside monomer and myricetin-3-O-(4″-galloyl)-α-L-rhamnoside monomer), as shown in Example 16 and FIG. 9.

The volume percentage of the alcohol solution is 50-100%, as the target products have poor water solubility but strong lipophilicity. Therefore, using an alcohol solution with a low volume concentration would reduce the extraction efficiency of the target products.

Preferably, the mass-to-volume ratio of bayberry leaves to methanol solution is 1:5 to 1:20. A ratio that is too low results in insufficient extraction, while a ratio that is too high leads to unnecessary reagent waste.

Preferably, the ultrasound extraction time is 30-60 minutes. An extraction time that is too short is insufficient, while an overly long time causes the temperature of the extraction solution to rise, affecting the extraction outcome. The extraction is preferably repeated 2 to 4 times; too few extractions fail to fully extract the target components, while too many result in solvent waste.

The invention also provides the use of myricetin-3-O-(2″-galloyl)-α-L-rhamnoside as an active ingredient in the preparation of α-glucosidase inhibitors; preferably, the myricetin-3-O-(2″-galloyl)-α-L-rhamnoside is separated and purified by any of the methods described previously.

The invention also provides the use of myricetin-3-O-(4″-galloyl)-α-L-rhamnoside as an active ingredient in the preparation of α-glucosidase inhibitors, preferably, the myricetin-3-O-(4″-galloyl)-α-L-rhamnoside is separated and purified by any of the methods described previously.

Preferably, the α-glucosidase inhibitors are used as therapeutic agents or medications for improving metabolic syndrome; preferably, the metabolic syndrome is at least one of type 2 diabetes, obesity, insulin resistance, hyperinsulinemia; preferably, the therapeutic agent is selected from functional foods, food additives, dietary supplements.

The pharmaceutical compositions of the present invention, in addition to containing the aforementioned myricetin-3-O-(2″-galloyl)-α-L-rhamnoside and/or myricetin-3-O-(4″-galloyl)-α-L-rhamnoside, may also include other active ingredients and pharmaceutically acceptable additives. Specific dosage forms for the pharmaceutical compositions of the invention include, but are not limited to, tablets, granules (including powders), capsules, and liquid formulations (including syrups). It is appropriate to use additives or bases suitable for each dosage form, manufactured according to conventional methods recorded in pharmacopoeias. The route of administration is not particularly limited and can include oral and non-oral administration. Examples of non-oral administration include buccal, tracheal, rectal, subcutaneous, intramuscular, and intravenous administration.

The metabolic syndrome improvers of the present invention may also contain various additives and other supplements, such as other active ingredients, vitamins (including Vitamin C), amino acids, oligosaccharides, etc. The form of the improvers is not particularly limited and can include tablets, granules (including powders), capsules, and liquid formulations (including syrups).

The functional foods of the present invention may also contain various additives, such as other active ingredients. The form of the functional foods is not particularly limited and can include noodles, pastries, and functional beverages.

The food additives of the present invention may also contain various additives, such as other active ingredients. The form of the food additives is not particularly limited and can include liquid, paste, powder, flake, and granular forms. Additionally, the food additives of the present invention include those used in beverages.

Further activity testing has found that the purified myricetin-3-O-(2″-galloyl)-α-L-rhamnoside monomer significantly inhibits α-glucosidase with an IC₅₀ value of 1.32 μM, which is significantly better than the positive control drug acarbose (IC₅₀=369.15 μM). It can serve as a new type of naturally sourced α-glucosidase inhibitor for controlling postprandial blood sugar and preventing and treating metabolic syndromes such as diabetes, obesity, and insulin resistance.

Further activity testing has revealed that the myricetin-3-O-(4″-galloyl)-α-L-rhamnoside monomer, obtained through purification, exhibits significant inhibition against α-glucosidase with an IC₅₀ value of 1.77 μM, which is notably superior to the positive control drug acarbose (IC₅₀=369.15 μM). This makes it a promising candidate as a new type of naturally sourced α-glucosidase inhibitor. It can be utilized to control postprandial blood glucose levels and prevent and treat metabolic syndromes such as diabetes, obesity, and insulin resistance.

### Advantages of the Invention Over Existing Technology

Novel Isolation and Purification: This invention pioneers the separation and purification of myricetin-3-O-(2″-galloyl)-α-L-rhamnoside monomer and myricetin-3-O-(4″-galloyl)-α-L-rhamnoside monomer from bayberry leaves. The structural similarity of these two flavonoid monomers has posed challenges for existing techniques, which the invention has overcome by providing a simple and efficient method for their purification.

Accessibility of Raw Materials: The starting material is readily available from bayberry leaves. The separation process is simple, quick, environmentally friendly, and yields highly pure monomers. This also serves as a reference for the separation and purification of other plant active substances, which is highly significant for the study of effective components in natural products.

The compounds isolated in this invention, myricetin-3-O-(2″-galloyl)-α-L-rhamnoside and myricetin-3-O-(4″-galloyl)-α-L-rhamnoside, possess various pharmacological activities, including antioxidant properties. Their significant inhibition of α-glucosidase activity makes them valuable for the preparation of α-glucosidase inhibitors. The research and further development of bayberry leaves are of great importance due to these findings.

### BRIEF DESCRIPTION OF THE DRAWINGS

FIG. 1 illustrates the high-performance liquid chromatography (HPLC) profile of the crude flavonol extract from bayberry leaves in Example 1. In this figure, “2″” represents the myricetin-3-O-(2″-galloyl)-α-L-rhamnoside monomer, and “4″” represents the myricetin-3-O-(4″-galloyl)-α-L-rhamnoside monomer.

FIG. 2 displays the HPLC profile of the solid-phase extraction powder rich in the target products from Example 1. Here, 2″ denotes the myricetin-3-O-(2″-galloyl)-α-L-rhamnoside monomer, and 4″ denotes the myricetin-3-O-(4″-galloyl)-α-L-rhamnoside monomer.

FIG. 3 is the HPLC chromatogram of myricetin-3-O-(2″-galloyl)-α-L-rhamnoside monomer (a) and myricetin-3-O-(4″-galloyl)-α-L-rhamnoside monomer (b) finally separated and purified in Example 1.

FIG. 4 is the HPLC chromatogram of myricetin-3-O-(2″-galloyl)-α-L-rhamnoside monomer (a) and myricetin-3-O-(4″-galloyl)-α-L-rhamnoside monomer (b) finally separated and purified in Example 2.

FIG. 5 depicts the high-performance liquid chromatography (HPLC) chart of the final purified myricetin-3-O-(2″-galloyl)-α-L-rhamnoside monomer (a) and myricetin-3-O-(4″-galloyl)-α-L-rhamnoside monomer (b) obtained from the separation process in Example 3.

FIG. 6A presents the primary ion fragmentation pattern of liquid chromatography-mass spectrometry (LC-MS) identification charts for the myricetin-3-O-(2″-galloyl)-α-L-rhamnoside monomer.

FIG. 6B presents secondary ion fragmentation pattern of liquid chromatography-mass spectrometry (LC-MS) identification charts for the myricetin-3-O-(2″-galloyl)-α-L-rhamnoside monomer.

FIG. 6C presents the nuclear magnetic resonance (NMR) identification spectrum of liquid chromatography-mass spectrometry (LC-MS) identification charts for the myricetin-3-O-(2″-galloyl)-α-L-rhamnoside monomer.

FIG. 7A illustrates primary ion fragmentation pattern of LC-MS identification charts for the myricetin-3-O-(4″-galloyl)-α-L-rhamnoside monomer.

FIG. 7B illustrates secondary ion fragmentation pattern of LC-MS identification charts for the myricetin-3-O-(4″-galloyl)-α-L-rhamnoside monomer.

FIG. 7C illustrates the NMR identification spectrum of LC-MS identification charts for the myricetin-3-O-(4″-galloyl)-α-L-rhamnoside monomer.

FIG. 8 displays the α-glucosidase inhibitory activity curves for the purified myricetin-3-O-(2″-galloyl)-α-L-rhamnoside monomer and myricetin-3-O-(4″-galloyl)-α-L-rhamnoside monomer. For comparative analysis, the figure also includes the α-glucosidase inhibitory activity curve for acarbose, a known inhibitor.

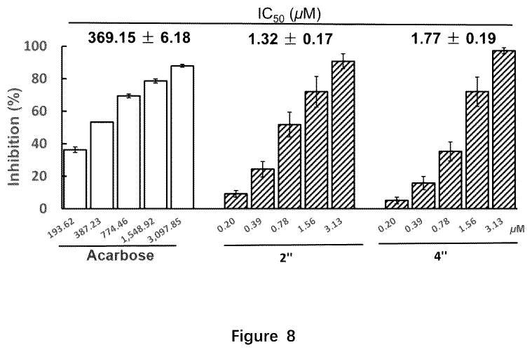

FIG. 9 shows the HPLC detection chromatograms of bayberry leaves extracted with solvents of different polarities.

### DETAILED DESCRIPTION OF THE EMBODIMENTS

The following provides further descriptions of the present invention with specific examples. The examples given are only illustrative and the scope of the invention is not limited to these:

### Example 1

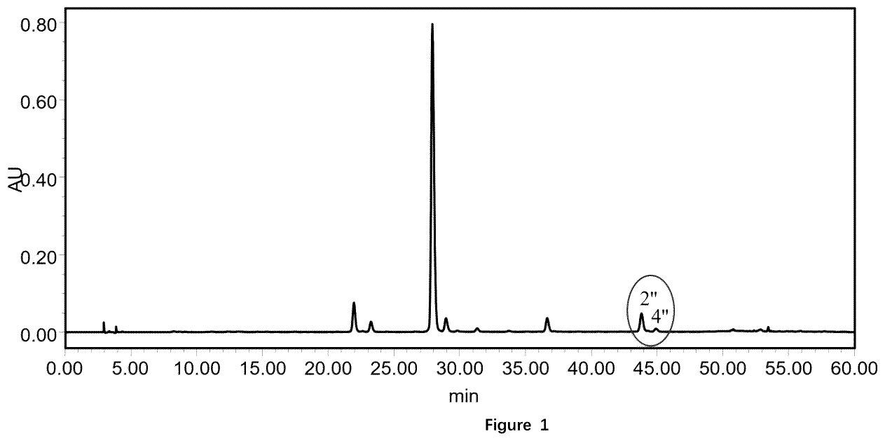

Weigh 20 g of bayberry leaves and add pure methanol solution according to the ratio of 1:10 (w/v, g/mL) to fully mix. Ultrasonically extract for 30 minutes, then filter after the ultrasound ends. The residue is re-extracted under the same conditions, and the filtrates are combined. Evaporate the methanol under vacuum rotary evaporation at 40° C. to obtain the flavonol crude extract (FIG. 1).

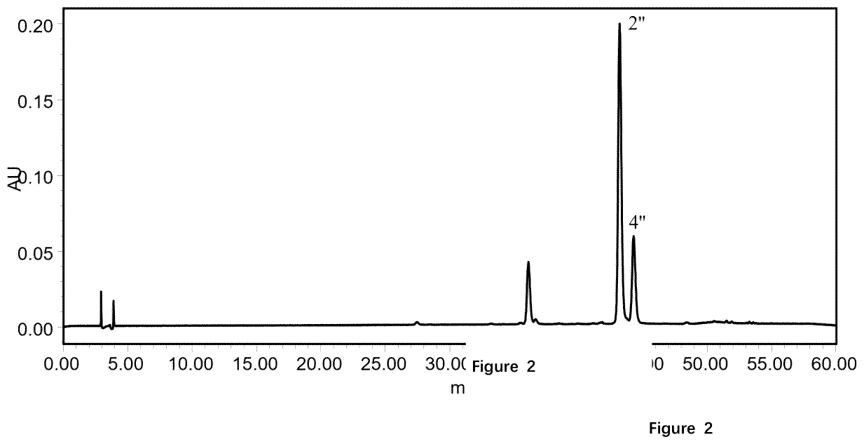

First, activate the C18 Sep-Pak® solid-phase extraction cartridge (Waters 12 cc, 2 g) with 4 bed volumes (BV) of methanol and 2 BV of water. Then, load the flavonol crude extract onto each solid-phase extraction cartridge using 4.5 BV; wash away saccharic acid with 4 BV of deionized water; elute with 10 BV of a 30% methanol solution and 4 BV of a 40% methanol solution, collecting the 40% methanol solution eluate. Dry the collected eluate under vacuum rotary evaporation at 40° C. to obtain the solid-phase extraction powder rich in the target product (FIG. 2).

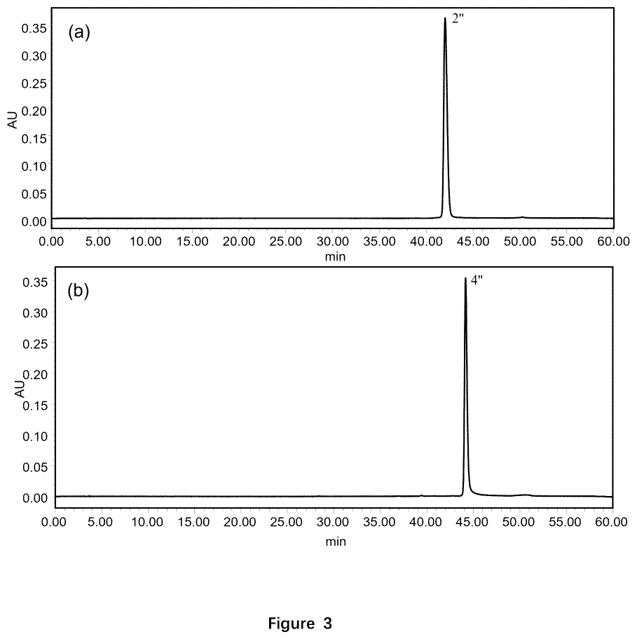

Utilize a preparative liquid chromatography column, SunFire™ C18 OBM™ (5 μm, 19×250 mm), with the mobile phase consisting of: Phase A, a 0.1% aqueous solution of formic acid, and Phase B, a 50% acetonitrile solution containing 0.1% formic acid; the column temperature is set at 25° C., with a flow rate of 5 mL/min, and the gradient elution is as follows: 0-10 min, 20%-60% B; 10-30 min, 60%-90% B; 30-35 min, 90%-100% B; 35-40 min, 100%-20% B. Dissolve the solid-phase extraction powder in methanol to a concentration of 100 mg/mL, inject it into the preparative liquid chromatography for separation, with a single injection volume of 100 μL. Detected under Waters 2998 PAD detector, collected the eluates for 27˜28.5 min and 28.5-30 min respectively, concentrated under reduced pressure, and freeze-dried in vacuum to obtain myricetin-3-O-(2″-galloyl)-α-L-rhamnoside monomer powder with a purity of 98.86% (FIG. 3, the upper part), and the myricetin-3-O-(4″-galloyl)-α-L-rhamnoside monomer powder with a purity of 99% (FIG. 3, the lower part).

### Example 2

Weigh 30 g of bayberry leaves and add an 80% methanol water solution (with a methanol to water volume ratio of 80:20) according to the ratio of 1:10 (w/v, g/mL) to fully mix. Ultrasonically extract for 30 minutes, then filter after the ultrasound ends. The residue is re-extracted under the same conditions, and the filtrates are combined. Evaporate the methanol under vacuum rotary evaporation at 37° C. to obtain the flavonol crude extract.

Activate the C18 Sep-Pak® solid-phase extraction cartridge with 4 bed volumes (BV) of methanol and 2 BV of water, then load the flavonol crude extract onto each cartridge using 4.5 BV Wash away saccharic acid with 4 BV of deionized water. Elute with 10 BV of a 30% methanol solution and 4 BV of a 40% methanol solution, collecting the 40% methanol solution eluate. Dry the collected eluate under vacuum rotary evaporation at 37° C. to obtain the solid-phase extraction powder rich in the target product.

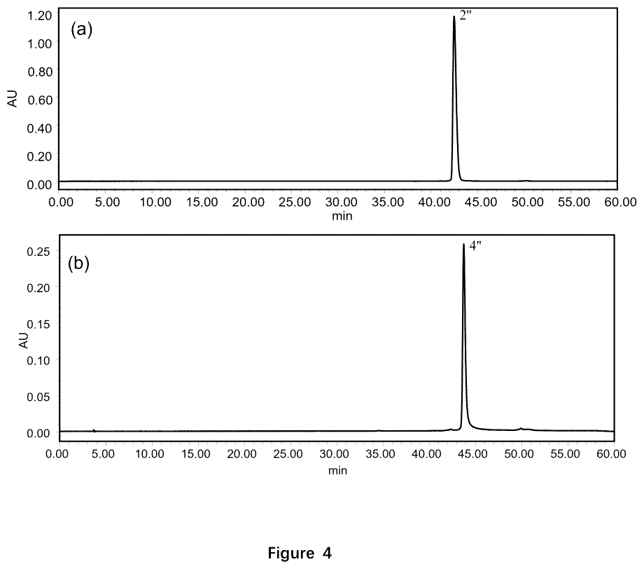

Utilize a preparative liquid chromatography column, SunFire™ C18 OBM™ (5 μm, 19×250 mm), with the mobile phase consisting of: Phase A, a 0.1% aqueous solution of formic acid, and Phase B, a 50% acetonitrile solution containing 0.1% formic acid; the column temperature is set at 25° C., with a flow rate of 5 mL/min, and the gradient elution is as follows: 0-10 min, 20%-60% B; 10-30 min, 60%-90% B; 30-35 min, 90%-100% B; 35-40 min, 100%-20% B. Dissolve the solid-phase extraction powder in methanol to a concentration of 150 mg/mL, inject it into the preparative liquid chromatography for separation, with a single injection volume of 150 μL. Detect under a Waters 2998 PAD detector, and collect the eluates separately during 27-28.5 min and 28.5-30 min. After concentration under reduced pressure and vacuum freeze-drying, obtain the myricetin-3-O-(2″-galloyl)-α-L-rhamnoside monomer powder with a purity of 98.19% (FIG. 4, the upper part), and the myricetin-3-O-(4″-galloyl)-α-L-rhamnoside monomer powder with a purity of 98.32% (FIG. 4, the lower part).

### Example 3

Weigh 60 g of bayberry leaves and add an 80% methanol water solution (with a methanol to water volume ratio of 80:20) according to the ratio of 1:10 (w/v, g/mL) to fully mix. Ultrasonically extract for 30 minutes, then filter after the ultrasound ends. The residue is re-extracted under the same conditions, and the filtrates are combined. Evaporate the methanol under vacuum rotary evaporation at 37° C. to obtain the flavonol crude extract.

Activate the C18 Sep-Pak® solid-phase extraction cartridge with 4 bed volumes (BV) of methanol and 2 BV of water, then load the flavonol crude extract onto each cartridge using 4.5 BV Wash away saccharic acid with 4 BV of deionized water. Elute with 10 BV of a 30% methanol solution and 4 BV of a 40% methanol solution, collecting the 40% methanol solution eluate. Dry the collected eluate under vacuum rotary evaporation at 37° C. to obtain the solid-phase extraction powder rich in the target product.

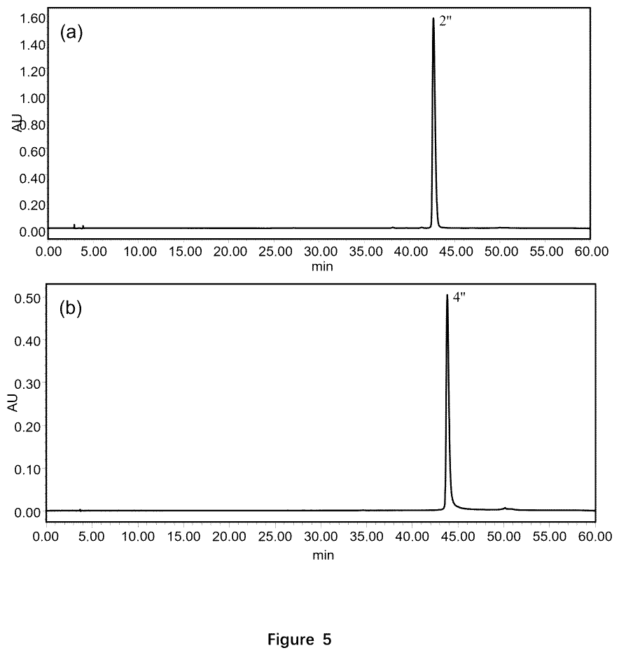

Utilize a preparative liquid chromatography column, SunFire™ C18 OBM™ (5 μm, 19×250 mm), with the mobile phase consisting of: Phase A, pure water containing 0.1% formic acid, and Phase B, 50% acetonitrile containing 0.1% formic acid; the column temperature is set at 25° C., with a flow rate of 5 mL/min, and the gradient elution is as follows: 0-10 min, 20%-60% B; 10-30 min, 60%-90% B; 30-35 min, 90%-100% B; 35-40 min, 100%-20% B. Dissolve the powder rich in the target product in methanol to a concentration of 150 mg/mL, inject it into the preparative liquid chromatography for separation, with a single injection volume of 200 μL. Detect under a Waters 2998 PAD detector, and collect the eluates separately during 27-28.5 min and 28.5-30 min. After concentration under reduced pressure and vacuum freeze-drying, obtain the myricetin-3-O-(2″-galloyl)-α-L-rhamnoside monomer powder with a purity of 98.30% (FIG. 5, the upper part), and the myricetin-3-O-(4″-galloyl)-α-L-rhamnoside monomer powder with a purity of 98.13% (FIG. 5, the lower part).

Following the separation method of Example 1, modify some parameters in the process to carry out the following Examples 4 to 13 (Note: The concentrations in the table are by volume percent, the 2″ monomer refers to myricetin-3-O-(2″-galloyl)-α-L-rhamnoside monomer; the 4″ monomer refers to myricetin-3-O-(4″-galloyl)-α-L-rhamnoside monomer).

| The various | | | | | | |
|---|---|---|---|---|---|---|
| embodiments | Example 4 | Example 5 | Example 6 | Example 7 | Example 8 | Example 9 |
| The mass-to- | 1:5 | 1:20 | 1:10 | 1:15 | 1:5 | 1:20 |
| volume ratio of | | | | | | |
| bayberry leaves | | | | | | |
| to methanol | | | | | | |
| solution | | | | | | |
| Alcohol | Methanol | Ethanol | Methanol | Ethanol | Methanol | Ethanol |
| The volume | 50% | 60% | 80% | 80% | 90% | 100% |
| percentage | | | | | | |
| concentration | | | | | | |
| of the alcohol | | | | | | |
| solution in | | | | | | |
| the alcohol | | | | | | |
| extraction and | | | | | | |
| concentration | | | | | | |
| process | | | | | | |
| The concentration | 20% | 25% | 30% | 35% | 38% | 20% |
| of the alcohol | | | | | | |
| solution for the | | | | | | |
| first elution | | | | | | |
| The concentration | 40% | 45% | 40% | 55% | 60% | 40% |
| of the alcohol | | | | | | |
| solution for the | | | | | | |
| second elution | | | | | | |
| Ultrasound | 30 | 40 | 30 | 60 | 30 | 40 |
| extraction time | | | | | | |
| Phase A | 0.05% formic | 0.05% tri- | 0.1% formic | 5% tri- | 3% formic | 3% tri- |
| | acid-water | fluoroacetic | acid-water | fluoroacetic | acid-water | fluoroacetic |
| | solution | acid-water | solution | acid-water | solution | acid-water |
| | | solution | | solution | | solution |
| Phase B | 40% ace- | 60% ace- | 50% formic | 45% formic | 55% ace- | 55% formic |
| | tonitrile- | tonitrile- | acid-ace- | acid-ace- | tonitrile- | acid-ace- |
| | water | water | tonitrile- | tonitrile- | water | tonitrile- |
| | solution | solution | water | water | solution | water |
| | | | solution; | solution; | | solution; |
| | | | The volume | The volume | | The volume |
| | | | percentage | percentage | | percentage |
| | | | concentration | concentration | | concentration |
| | | | of formic acid | of formic acid | | of formic acid |
| | | | is 0.05% | is 3% | | is 0.1% |
| 2″ monomer | 98.31% | 98.25% | 99.12% | 98.55% | 98.56% | 98.64% |
| purity | | | | | | |
| 4″ monomer | 98.09% | 98.18% | 99.2% | 98.20% | 98.44% | 98.52% |
| purity | | | | | | |

| The various | | | | |
|---|---|---|---|---|
| embodiments | Example 10 | Example 11 | Example 12 | Example 13 |
| The mass-to- | 1:10 | 1:15 | 1:5 | 1:20 |
| volume ratio of | | | | |
| bayberry leaves | | | | |
| to methanol | | | | |
| solution | | | | |
| Alcohol | Methanol | Ethanol | Methanol | Ethanol |
| The volume | 60% | 70% | 80% | 60% |
| percentage | | | | |
| concentration | | | | |
| of the alcohol | | | | |
| solution in | | | | |
| the alcohol | | | | |
| extraction and | | | | |
| concentration | | | | |
| process | | | | |
| The concentration | 25% | 30% | 35% | 38% |
| of the alcohol | | | | |
| solution for the | | | | |
| first elution | | | | |
| The concentration | 45% | 50% | 55% | 60% |
| of the alcohol | | | | |
| solution for the | | | | |
| second elution | | | | |
| Ultrasound | 50 | 60 | 30 | 40 |
| extraction time | | | | |
| Phase A | 0.1% formic | 0.1 tri- | 1% formic | 1% tri- |
| | acid-water | fluoroacetic | acid-water | fluoroacetic |
| | solution | acid-water | solution | acid-water |
| | | solution | | solution |
| Phase B | 45% ace- | 45% tri- | 50% ace- | 50% tri- |
| | tonitrile- | fluoroacetic | tonitrile- | fluoroacetic |
| | water | acid- | water | acid-ace- |
| | solution | acetonitrile- | solution | tonitrile- |
| | | water | | water |
| | | solution; | | solution; |
| | | The volume | | The volume |
| | | percentage | | percentage |
| | | concentration | | concentration |
| | | of tri- | | of tri- |
| | | fluoroacetic | | fluoroacetic |
| | | acid is 0.05% | | acid is 5% |
| 2″ monomer | 98.69% | 98.63% | 98.47% | 98.47% |
| purity | | | | |
| 4″ monomer | 98.47% | 98.22% | 98.59% | 98.14% |
| purity | | | | |

### Example 14

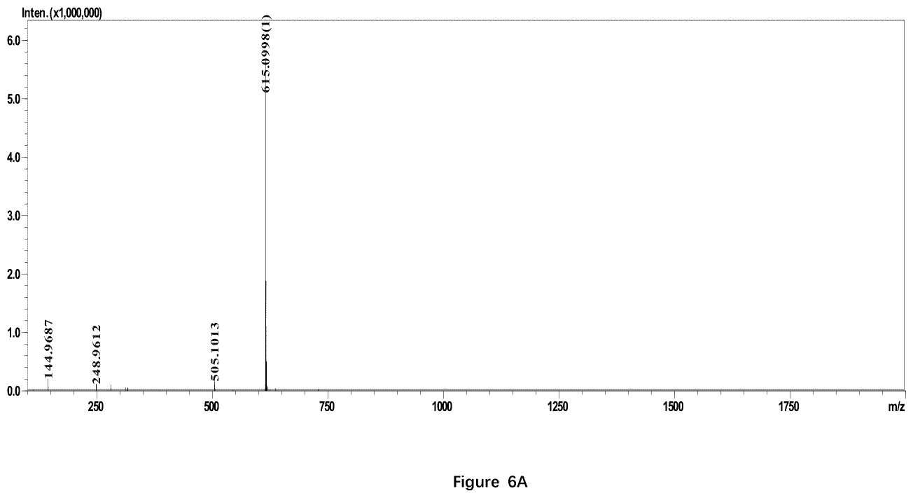

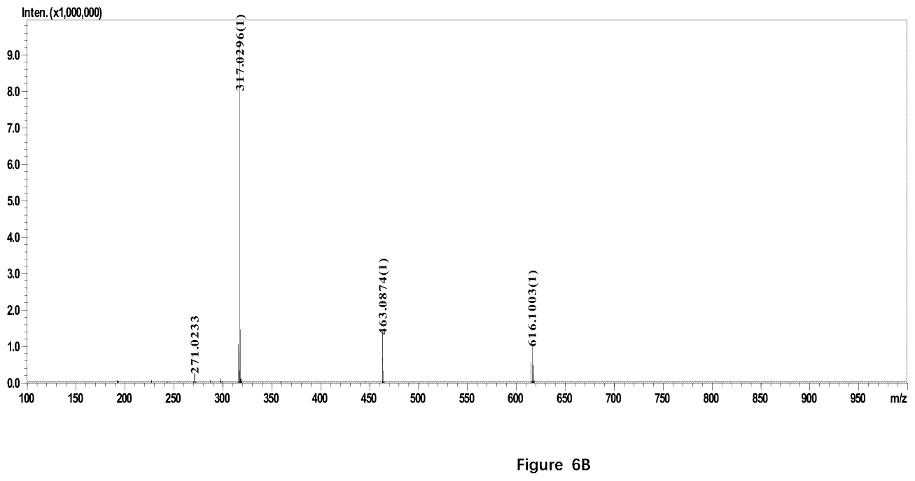

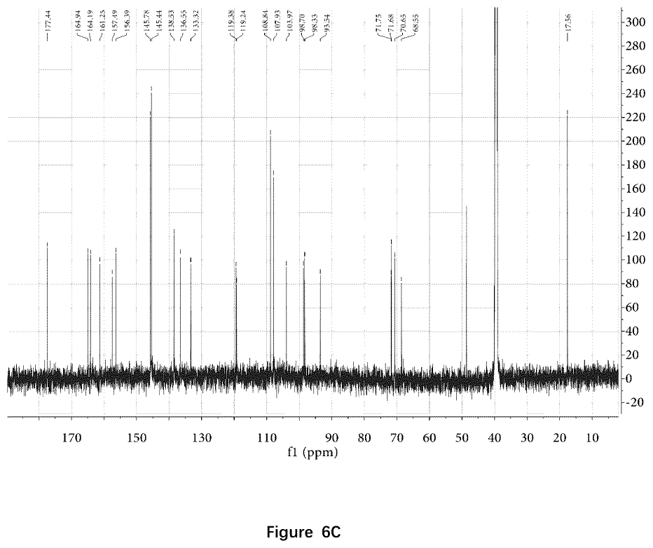

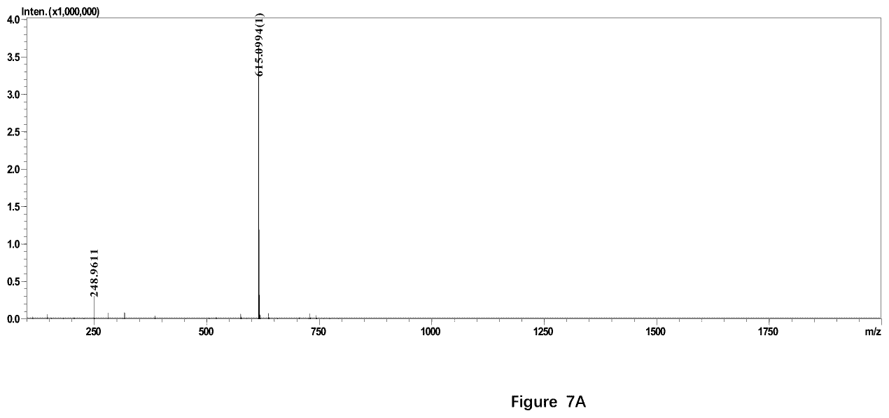

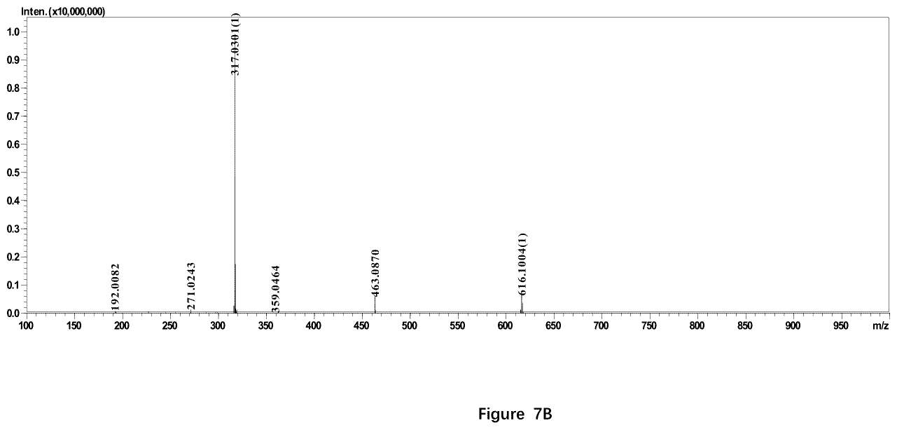

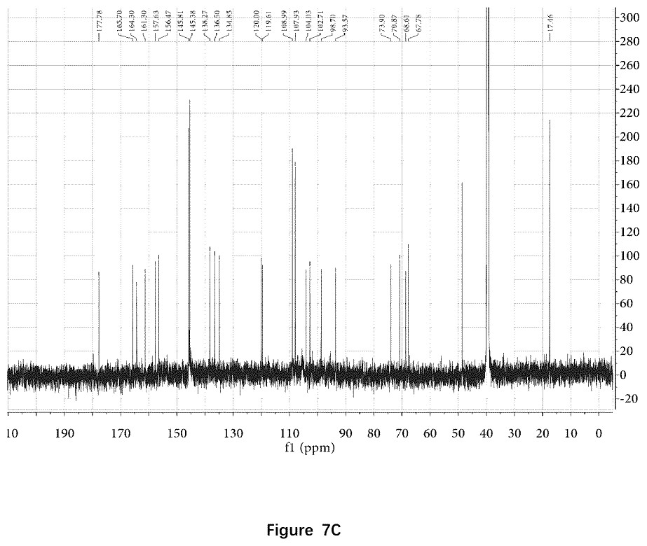

The structures of the two compound monomers separated from the aforementioned three examples were analyzed using high-resolution LC-MS and NMR techniques. The primary and secondary ion fragmentation patterns identified by high-resolution LC-MS, as well as the NMR identification spectra, are shown in FIGS. 6A-6C and FIGS. 7A-7C of the appendix. It was thereby determined that the two purified monomers are isomers, specifically myricetin-3-O-(2″-galloyl)-α-L-rhamnoside and myricetin-3-O-(4″-galloyl)-α-L-rhamnoside.

Myricetin-3-O-(2″-galloyl)-α-L-rhamnoside. ¹³C NMR (151 MHz, DMSO-d6) δ 177.44 (C-4), 164.94 (COO), 164.19 (C-7), 161.25 (C-5), 157.49 (C-2), 156.39 (C-9), 145.78 (C-3′/5′), 145.44 (C-3′″/5′″), 138.53 (C-4′″), 136.55 (C-4′), 133.32 (C-3), 119.38 (C-1′), 119.24 (C-1′″), 108.84 (C-2′/6′), 107.93 (C-2′″/6′″), 103.97 (C-10), 98.70 (C-6), 98.33 (C-1″), 93.54 (C-8), 71.75 (C-4″), 71.68 (C-2″), 70.65 (C-5″), 68.55 (C-3″), 17.56 (C-6″).

Myricetin-3-O-(4″-galloyl)-α-L-rhamnoside. ¹³C NMR (151 MHz, DMSO-d6) δ 177.78 (C-4), 165.70 (COO), 164.30 (C-7), 161.30 (C-5), 157.63 (C-2), 156.47 (C-9), 145.81 (C-3′/5′), 145.38 (C-3′″/5′″), 138.27 (C-4′″), 136.50 (C-4′), 134.85 (C-3), 120.00 (C-1′″), 119.61 (C-1′), 108.99 (C-2′/6′), 107.93 (C-2′″/6′″), 104.03 (C-10), 102.71 (C-1″), 98.70 (C-6), 93.57 (C-8), 73.90 (C-4″), 70.87 (C-2″), 68.61 (C-3″), 67.78 (C-5″), 17.46 (C-6″).

### Example 15

α-Glucosidase can hydrolyze 4-nitrophenyl β-D-glucopyranoside (PNPG) to produce p-nitrophenol (pNP), which has its maximum absorbance at 405 nm under alkaline conditions. The concentration of pNP in the reaction solution, as measured by a plate reader, allows for the detection of the sample's ability to inhibit the activity of α-glucosidase.

α-Glucosidase is diluted to 0.2 U/mL with 0.1 M buffer solution (pH=6.8). For the assay, a 96-well plate is loaded with 112 μL of phosphate buffer (0.1 M, pH=6.8) and 20 μL of α-glucosidase (0.2 U/mL), followed by the addition of 8 μL of the inhibitor, which is a gradient concentration of myricetin-3-O-(2″-galloyl)-α-L-rhamnoside monomer or myricetin-3-O-(4″-galloyl)-α-L-rhamnoside monomer or acarbose. The mixture is then incubated at 37° C. for 15 minutes, after which 20 μL of PNPG solution (2.5 mM) is added. After a 15-minute reaction at 37° C., 80 μL of Na₂CO₃ solution (2.5 mM) is added, and the absorbance at 405 nm (ODₜₑₛₜ) is measured using a plate reader. The blank control without enzyme is denoted as OD_{blank}, the control with enzyme but without the sample is controlODₜₑₛₜ, and the control without both the sample and enzyme is controlOD_{blank}, with acarbose serving as the positive control. The formula for calculating the enzyme activity inhibition rate is as follows:

$$α - Glucosidase inhibition rate ( \% ) = 1 - \frac{{OD}_{test} - {OD}_{blank}}{{controlOD}_{test} - {controlOD}_{blank}}$$

The inhibitory effect of the tested monomers on α-glucosidase a gradient concentrations was used to calculate the IC50 values with SPSS. The results indicated that both myricetin-3-O-(2″-galloyl)-α-L-rhamnoside monomer and myricetin-3-O-(4″-galloyl)-α-L-rhamnoside monomer significantly inhibited α-glucosidase, with IC₅₀ values of 1.32 μM and 1.77 μM, respectively, which are significantly better than the positive control drug acarbose (IC₅₀=369.15 μM). This suggests that myricetin-3-O-(2″-galloyl)-α-L-rhamnoside and myricetin-3-O-(4″-galloyl)-α-L-rhamnoside are potent alpha-glucosidase inhibitors and can be developed as therapeutic drugs for diabetes. They can be applied to improve insulin resistance and prevent and treat metabolic syndromes such as diabetes and obesity.

### Example 16

### Solvent Selection for Extraction

Weigh 0.1 g of freeze-dried bayberry leaf powder and dissolve it in 1 mL of five selected organic solvents. After ultrasonic extraction for 30 minutes, centrifuge at 10,000 rpm for 10 minutes and take the supernatant. Repeat the extraction process once more, and combine the two supernatants for HPLC analysis and detection.

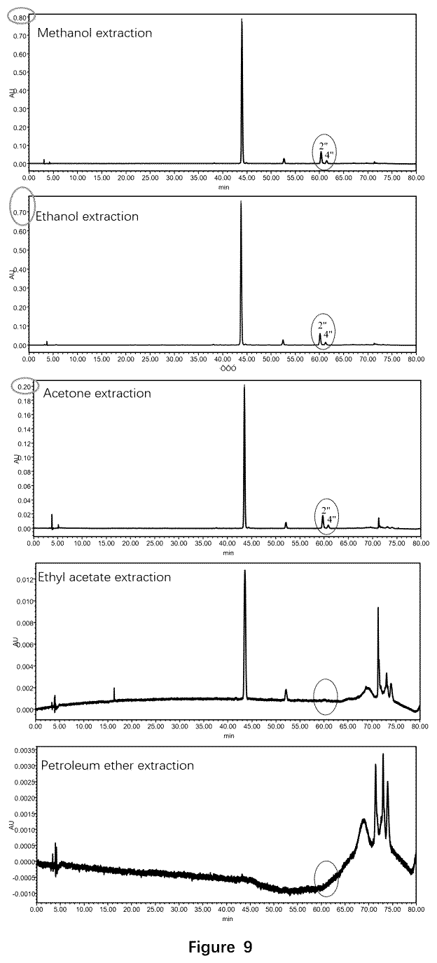

The relative content of the specified compounds in the solution with equal concentrations can be determined by the peak area detected by HPLC. As shown in FIG. 9, acetone extract has a low concentration of the target product and cannot fully extract the target product from the sample. The target products were not detected in the ethyl acetate and petroleum ether extracts. The target products refer to myricetin-3-O-(2″-galloyl)-α-L-rhamnoside monomer and myricetin-3-O-(4″-galloyl)-α-L-rhamnoside monomer. Therefore, methanol and ethanol are more suitable for the extraction of the two target products from bayberry leaves.

### Comparative Example 1

Weigh 20 g of bayberry leaves and add pure methanol solution according to the ratio of 1:10 (w/v, g/mL) to fully mix. Ultrasonically extract for 30 minutes, then filter after the ultrasound ends. The residue is re-extracted under the same conditions, and the filtrates are combined. Evaporate the methanol under vacuum rotary evaporation at 40° C. to obtain the flavonol crude extract.

Activate the C18 Sep-Pak® solid-phase extraction cartridge (Waters 12 cc, 2 g) with 4 bed volumes (BV) of methanol and 2 BV of water, then load the flavonol crude extract onto each cartridge using 4.5 BV Wash away saccharic acid with 4 BV of deionized water. Elute with 10 BV of a 10% methanol solution and 4 BV of a 40% methanol solution, collecting the 40% methanol solution eluate. Dry the collected eluate under vacuum rotary evaporation at 40° C. to obtain the solid-phase extraction powder rich in the target product.

Utilize a preparative liquid chromatography column, SunFire™ C18 OBM™ (5 μm, 19×250 mm), with the mobile phase consisting of: Phase A, a 0.1% aqueous solution of formic acid, and Phase B, a 50% acetonitrile solution containing 0.1% formic acid; the column temperature is set at 25° C., with a flow rate of 5 mL/min, and the gradient elution is as follows: 0-10 min, 20%-60% B; 10-30 min, 60%-90% B; 30-35 min, 90%-100% B; 35-40 min, 100%-20% B. Dissolve the solid-phase extraction powder in methanol to a concentration of 100 mg/mL, inject it into the preparative liquid chromatography for separation, with a single injection volume of 100 μL. Detect under a Waters 2998 PAD detector, and collect the eluates separately during 27-28.5 min and 28.5-30 min. After concentration under reduced pressure and vacuum freeze-drying, obtain the myricetin-3-O-(2″-galloyl)-α-L-rhamnoside monomer powder with a purity of 80.18%, and the myricetin-3-O-(4″-galloyl)-α-L-rhamnoside monomer powder with a purity of 75.25%.

### Comparative Example 2

Weigh 20 g of bayberry leaves and add pure methanol solution according to the ratio of 1:10 (w/v, g/mL) to fully mix. Ultrasonically extract for 30 minutes, then filter after the ultrasound ends. The residue is re-extracted under the same conditions, and the filtrates are combined. Evaporate the methanol under vacuum rotary evaporation at 40° C. to obtain the flavonol crude extract.

Activate the C18 Sep-Pak® solid-phase extraction cartridge (Waters 12 cc, 2 g) with 4 BV of methanol and 2 BV of water, then load the flavonol crude extract onto each cartridge using 4.5 BV. Wash away saccharic acid with 4 BV of deionized water. Elute with 10 BV of a 20% methanol solution and 4 BV of a 30% methanol solution, collecting the 30% methanol solution eluate. Dry the collected eluate under vacuum rotary evaporation at 40° C. and find that the obtained solid-phase extraction powder does not contain the target product.

### Comparative Example 3

Weigh 20 g of bayberry leaves and add petroleum ether solution according to the ratio of 1:10 (w/v, g/mL) to fully mix. Ultrasonically extract for 30 minutes, then filter after the ultrasound ends. The residue is re-extracted under the same conditions, and the filtrates are combined. It is found that the obtained crude extract does not contain the target product.

What is claimed is:

1. A method for simultaneously separating and purifying myricetin-3-O-(2″-galloyl)-α-L-rhamnoside and myricetin-3-O-(4″-galloyl)-α-L-rhamnoside from Bayberry leaves, comprising the following steps:
(1) alcohol extraction and concentration: mixing Bayberry leaves with an alcohol solution; extracting by ultrasound; filtering and collecting the filtrate; removing the alcohol from the filtrate and concentrating to obtain a crude Bayberry leaf flavonol alcohol extract; wherein the volume percentage of the alcohol solution is 50-100%;
(2) solid-phase extraction column adsorption: injecting the crude Bayberry leaf flavonol alcohol extract into a solid-phase extraction column; washing with a mobile phase for the first gradient elution; and processing the collected eluent to obtain a solid-phase extraction powder rich in myricetin-3-O-(2″-galloyl)-α-L-rhamnoside and myricetin-3-O-(4″-galloyl)-α-L-rhamnoside;
the first gradient elution process is as follows: first eluting with an alcohol solution with a volume percentage lower than 40% for the first elution; then using an alcohol solution with a volume percentage of 40-60% for the second elution; and collecting the eluent after the second elution; wherein the concentration of the alcohol solution for the first elution is above 20% by volume percentage;
(3) preparative liquid chromatography purification: using a solid-phase chromatography column; wherein the solid-phase extraction powder obtained from step (2) is subjected to a second gradient elution with a mobile phase and then processed to obtain the target products: myricetin-3-O-(2″-galloyl)-α-L-rhamnoside monomer and myricetin-3-O-(4″-galloyl)-α-L-rhamnoside monomer;
the mobile phase: Phase A is selected from a formic acid-water solution with a volume percentage of 0.05-5%, a trifluoroacetic acid-water solution with a volume percentage of 0.05-5%; Phase B is selected from an acetonitrile-water solution with a volume percentage of 40-60%, an acid-acetonitrile-water solution with a volume percentage of 40-60%; the volume percentage of the acid is 0.05-5%, and the acid is selected from formic acid, trifluoroacetic acid;
the second gradient elution process is as follows: the volume percentage of Phase B increases from 20% to 60% within 0-10 minutes, from 60% to 90% within 10-30 minutes, and from 90% to 100% within 30-35 minutes; and then decreases from 100% to 20% within 35-40 minutes, collecting the target products separately;
the alcohol solution refers to an alcohol aqueous solution, and the alcohol is selected from methanol or ethanol.

2. The method according to claim 1, wherein in step (1), the filtrate is concentrated by vacuum rotary evaporation at 37-50° C. to remove the alcohol and obtain the crude Bayberry leaf flavonol alcohol extract.

3. The method according to claim 1, wherein in step (1), the process of collecting the filtrate is repeated 2-4 times, and the filtrates of 2-4 times are combined.

4. The method according to the method of claim 1, wherein in step (2), the gradient elution of the solid phase extraction column is as follows:
the crude Bayberry leaf flavonol alcohol extract is injected into the solid-phase extraction column, washed with deionized water; and gradient elution is performed with a mobile phase; and the collected eluent is vacuum rotary evaporated at 37-45° C. to obtain the solid phase extraction powder rich in the target product.

5. The method according to the method of claim 1, wherein in step (2), the adsorption of the solid phase extraction column is as follows:
the C18 Sep-Pak® solid phase extraction column is activated, and 4.5 BV of Bayberry leaf extract is applied to each column; 4 BV of deionized water is used to wash away saccharic acid; then 10 BV of a 30% methanol solution is used to elute, removing some impurities and other non-target flavonols; then 4 BV of a 40% methanol solution is used to elute, collecting the eluent of the 40% fraction, and vacuum rotary evaporated at 37-50° C. to obtain the solid phase extraction powder rich in myricetin-3-O-(2″-galloyl)-α-L-rhamnoside and myricetin-3-O-(4″-galloyl)-α-L-rhamnoside.

6. The method according to claim 1, wherein in step (3), Phase B is selected from a 50% acetonitrile-water solution, a 50% acid-acetonitrile-water solution; the volume percentage of the acid is 0.1-5%; and the acid is selected from formic acid, trifluoroacetic acid.

7. The method according to claim 1, wherein in step (3), the preparative liquid chromatography purification is as follows:
a preparative liquid column SunFire™ C18 OBM™ column (5 μm, 19×250 mm) is used; the mobile phase: Phase A: a 0.1% formic acid-water solution, Phase B: a 50% formic acid-acetonitrile-water solution (wherein, the volume percentage of formic acid is 0.1%); the column temperature is room temperature, and the flow rate is 3-6 mL/min;
the solid phase extraction powder obtained in step (2) is dissolved in methanol to a concentration of 100-200 mg/mL, injected into the preparative liquid phase for purification, with a single injection volume of 50-300 μL;
the eluate is collected separately at 27-28.5 minutes and 28.5-30 minutes, and the eluate rich in pure target product is combined, and then concentrated and freeze-dried to obtain high purity myricetin-3-O-(2″-galloyl)-α-L-rhamnoside monomer and myricetin-3-O-(4″-galloyl)-α-L-rhamnoside monomer.

8. The method according to claim 1, wherein 30%-40% alcohol solution is used for the first elution.

9. The method according to claim 1, wherein Phase A is selected from a formic acid-water solution with a volume percentage of 0.1-3%.

10. The method according to claim 1, wherein in step (3), the preparative liquid column SunFire™ C18 OBM™ column (5 μm, 19×250 mm) is used; and during the second gradient elution, the eluate is collected separately at 27-28.5 minutes and 28.5-30 minutes; the column temperature is room temperature, and the flow rate is 3-6 mL/min.

11. The method according to claim 1, wherein the solid phase extraction powder obtained in step (2) is dissolved in methanol to a concentration of 100-200 mg/mL, injected into the preparative liquid phase for purification, with a single injection volume of 50-300 μL.

12. The method according to claim 1, wherein the volume percentage of the alcohol solution is 80%.

13. The method according to claim 1, wherein the ultrasound extraction time is 30-60 minutes.

14. The method according to claim 1, wherein the mass-to-volume ratio of Bayberry leaves to methanol solution is 1:5-20.

15. The method according to claim 14, wherein the mass-to-volume ratio of Bayberry leaves to methanol solution is 1:10.

16. The method according to claim 1, wherein the post-treatment refers to reduced pressure concentration and freeze-drying; the reduced pressure concentration is performed under vacuum rotary evaporation at 37-50° C.

17. The method according to claim 1, wherein the purity of myricetin-3-O-(2″-galloyl)-α-L-rhamnoside monomer and myricetin-3-O-(4″-galloyl)-α-L-rhamnoside monomer is above 98%.

18. The method according to claim 1, wherein the solid phase extraction column is a C18 solid phase extraction column.

19. The method according to claim 6, wherein the volume percentage of the acid is 0.1-3%.

20. The method according to claim 8, wherein 30% alcohol solution is used for the first elution.
